(19) United States
(12) Patent Application Publication
(10) Pub. No.: US 2026/0242413 A1
(43) Pub. Date: Aug. 20, 2026

(54) ENDOTOXIN NEUTRALISING AGENT

(71) Applicant: AMFERIA AB, Mölndal (SE)

(72) Inventors: Anand KUMAR RAJESKHARAN, Göteborg (SE); Saba ATEFYEKTA, Göteborg (SE); Martin ANDERSSON, Mölndal (SE); Edvin BLOMSTRAND, Hällingsjö (SE)

(73) Assignee: AMFERIA AB, Mölndal (SE)

(21) Appl. No.: 19/150,858

(22) PCT Filed: Feb. 19, 2024
(86) PCT No.: PCT/SE2024/050168

§ 371 Date: Jul. 24, 2025

(30) Foreign Application Priority Data
Feb. 21, 2023 (SE) 2330094-0

(51) Int. Cl. C07K 7/06; A61K 38/00; A61K 47/69; A61P 39/00; C07K 7/08

(52) U.S. Cl. CPC C07K 7/06; A61K 47/6903; A61P 39/00; C07K 7/08; A61K 38/00

(57) ABSTRACT

A peptide for use as an endotoxin neutralising agent. The peptide comprises an amino acid sequence having at least 80% sequence identity to the sequence RRPRPRPRP and at least four tryptophan residues provided at the C- or N-terminus or therebetween. A method of endotoxin neutralisation in vitro is provided. The method comprises contacting a patient sample with the peptide. An endotoxin neutralising amphiphilic hydrogel is also provided.

### FIELD OF THE INVENTION

The present disclosure relates to the endotoxin neutralising agents, specifically it relates to the use of peptides as an endotoxin neutralising agents.

### BACKGROUND OF THE INVENTION

Bacterial infections remain a threat to human health. This is partly due to an increase in bacterial resistance, demographic change as populations age, and a lack of newly approved antibiotic treatments.

Severe bacterial infections may lead to sepsis which is characterised by an inflammatory state affecting a patient systemically. This severe inflammatory state has a high morbidity and lethality.

A problem in treating sepsis is that a drug must be able to kill the bacteria without releasing the inflammation inducing toxins. In the case of gram-negative bacteria these inflammation inducing toxins are lipopolysaccharides (LPS), and in the case of gram-positive bacteria they are lipoproteins (LP/LPs).

WO 2019/074422 A1 (Amferia AB) describes an antimicrobial hydrogel and an antimicrobial peptide. The antimicrobial peptide was shown to have significant antimicrobial efficacy. However, the antimicrobial efficacy of an antimicrobial peptide does not necessarily correspond to its anti-endotoxin efficacy. There is no simple model for either antimicrobial efficacy or anti-endotoxin efficacy. Neither is there a clear correlation between fold-structure, length, and sequence and anti-endotoxin efficacy. (Brandenburg, K et al. Peptides with dual mode of action: Killing bacteria and preventing endotoxin-induced sepsis, Biochimica et Biophysica Acta. 2016, vol. 1858, pp. 971-979)

Antimicrobial peptides which are suitable for killing bacteria, and therein may considered as a potential treatment for sepsis along with numerous other conditions arising from bacterial infection, may, however be unsuitable in practice due to release of inflammation inducing toxins on killing of bacteria.

As free antimicrobial peptides have been shown to degrade rapidly in e.g., blood, the provision of antimicrobial peptides covalently attached to a substrate is an ideal wound treatment device. However, due to the changes in structure, available binding sites etc. the attachment of an antimicrobial peptide to a substrate is considered likely to negatively impact the anti-endotoxin effect of an effective antimicrobial peptide. It has been shown that AMP efficacy is reduced by 500-1000 times on immobilisation on surfaces (The Potential of Surface-Immobilized Antimicrobial Peptides for the Enhancement of Orthopaedic Medical Devices: A Review. Antibiotics (Basel). 2023; 12 (2): 211. 19 Jan. 2023), therefore endotoxin binding efficacy can also be expected to be reduced on immobilisation of an AMP to a surface.

Improved anti-endotoxin treatments would be advantageous. In particular antimicrobial peptides with combined anti-microbial and anti-endotoxin efficacy would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing an amphiphilic peptide for use as an endotoxin neutralising agent, the peptide comprising an amino acid sequence having at least 80% sequence identity to the sequence RRP9 (SEQ ID: 1), and at least four tryptophan residues provided at the C- or N-terminus or therebetween.

An amphiphilic hydrogel composition for use in endotoxin neutralisation is provided.

Furthermore, a method of endotoxin neutralisation in vitro is also provided.

Further advantageous embodiments are disclosed in the appended and dependent patent claims.

### BRIEF DESCRIPTION OF THE DRAWINGS

These and other aspects, features and advantages of which the invention is capable will be apparent and elucidated from the following description of embodiments of the present invention, reference being made to the accompanying drawings, in which:

FIG. 1 shows the results from the endotoxin neutralisation test as described in experiment 1 below. The endotoxin levels were detected after 3 hours and 24 hours. The left axis and left column in each pair of columns is the endotoxin level after 3 hours, and the right axis and right column in each pair is the endotoxin level after 24 hours.

FIG. 2 shows the results from the endotoxin neutralisation test as described in experiment 2 detailed below. The endotoxin levels were detected after 1 hour, and after 3 hours. AMP is the antimicrobial peptide RRP9W4N in solution. AMP-P is the same antimicrobial peptide bound to crosslinked hydrogel particles.

FIG. 3 shows the results from the endotoxin neutralisation test as described in experiment 2 detailed below. The results shown are the detected endotoxin levels after 24 hours. C—P are crosslinked hydrogel particles with no antimicrobial attached. PBS −b refers to PBS without bacteria and PBS +b refers to PBS with bacteria. AMP is the antimicrobial peptide RRP9W4N in solution. AMP-P is the same antimicrobial peptide bound to crosslinked hydrogel particles.

### DETAILED DESCRIPTION

The present disclosure relates to the use of an antimicrobial peptide as an anti-endotoxin agent. An anti-endotoxin agent may be referred to as an endotoxin neutralising agent. Additionally, an anti-endotoxin agent may be referred to as an anti-inflammatory agent due to its inflammation inhibiting effect. The anti-endotoxin agent may be for use in the treatment of bacterial sepsis. The endotoxin neutralising agent may be for use in vivo.

The antimicrobial peptide may be a proline arginine-rich end leucine-rich repeat protein, PRELP, derived antimicrobial peptide. The antimicrobial peptide may comprise a sequence having an 80%, such as 90%, such as 95%, identity to RRPRPRPRP (RRP9 SEQ ID: 1). Such peptides have been shown to be similar to human cathelicidin derived LL-37 with respect to several aspects, such as net charge and antimicrobial efficacy (Malmsten, M et al., Highly Selective End-Tagged Antimicrobial Peptides Derived from PRELP, PLOS ONE, 2011, 6 (1): e16400. doi: 10.1371/journal.pone.0016400).

The antimicrobial peptide is advantageously a short chain peptide consisting of from 1 to 50 amino acids, such as less than 40 amino acids, such as less than 30 amino acids, and preferably less than 20 amino acids. The antimicrobial peptide may have a molecular weight of between 1 to 50 kDa.

As stated above, the antimicrobial peptide may comprise a sequence having at least 90% identity to the sequence RRPRPRPRP (SEQ ID: 1, RRP9). The antimicrobial peptide ideally comprises a stretch of at least two consecutive tryptophan or phenylalanine residues appended to either the C- or N-terminus, or therebetween. The antimicrobial peptide may comprise a N-terminal amidation. The antimicrobial peptide is advantageously amphiphilic as this has previously been shown to have an improved effect and immobilisation properties.

The antimicrobial peptide may be for example RRPRPRPRPWWWW-NH2 (SEQ ID: 2, RRP9W4N), RRPRPRPRP-NH2 (SEQ ID: 3, RRP9N), RRPRPRPWWWWRP-NH2 (SEQ ID: 4, RRP7W4RPN), or RRPRPWWRPWWRP-NH2 (SEQ ID: 5, RRP5W2RPW2RPN). The above peptide sequences may be described as a peptide comprising the sequence RRP9 (SEQ ID: 1) and at least four tryptophan residues, the tryptophan residues being consecutive, or in at least two pairs of two consecutive residues separated by an arginine-proline pair, i.e., WWRPWW (SEQ ID: 6).

As shown in the experimental section, the antimicrobial peptide comprising a sequence corresponding to the sequences defined herein has been surprisingly shown to have an endotoxin neutralising effect. The antimicrobial peptides corresponding to the above definition has both cationic and hydrophobic parts, resulting in an amphiphilic antimicrobial peptide.

The peptide may be for use in methods of in vivo endotoxin neutralisation. A therapeutically effective amount of the peptide may be administered to a patient. The peptide may be for use in treating an acute inflammatory condition in a patient. The peptide may be for use in treating an endotoxin induced inflammatory condition. The peptide may be for use in treating LPS-induced inflammation. The inflammatory condition may be sepsis. Sepsis is defined as the presence or presumed presence of an infection accompanied by evidence of a systemic response called the systemic inflammatory response syndrome (SIRS). Sepsis is usually caused by bacterial infections, either gram-negative or gram-positive bacteria, but can also be caused by other pathogens such as viruses, fungi and protozoa. The injury and symptoms attributable to sepsis are not only caused by the bacteria but are also caused by a component of the bacteria cell wall known as endotoxin or LPS. LPS molecules are glycolipids that are ubiquitous in the outer membrane of Gram-negative bacteria. LPS is released when the immune system destroys the invading bacteria. The released LPS binds to monocytes, macrophages, and endothelial cells and triggers the production of various mediators such as TNF-a and interleukins (IL-1, IL-6, and IL-8). Production of excessive TNF-a, IL-1, IL-6, and IL-8 is a major cause of severe forms of sepsis.

The amount of the peptide which will be effective in the treatment of a particular disorder or condition will depend on the nature of the disorder or condition, and on the particular peptide. The effective amount can be determined by standard clinical techniques known to a person skilled in the art. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in vitro or in vivo animal model test bioassays or systems. A composition containing the peptide can be introduced, for example, into the systemic circulation, which will distribute said peptide within the patient.

The peptide may be used in vitro for endotoxin neutralisation. A patient sample, such as blood, plasma etc. of a patient may be brought into contact with the peptide, thereby neutralising endotoxins in the patient's blood etc. A method of in vitro endotoxin neutralisation may comprise contacting a patient sample, extracted from a patient, with the peptide such as RRP9W4N (SEQ ID: 2) or other sequences disclosed herein.

The antimicrobial peptide for use as an endotoxin neutralising agent may be attached, such as covalently attached, to a hydrogel substrate. The crosslinked hydrogel forms an ideal substrate to which the AMPs can be immobilised, as described above, when the peptide is used for in vitro endotoxin neutralisation.

As noted in WO 2019/074422 A1 (Amferia AB) if the hydrogel substrate is an amphiphilic hydrogel comprising repeating hydrophilic and hydrophobic regions the antimicrobial efficacy may be improved. However, as described above, improved antimicrobial efficacy does not correspond to an increased or even maintained anti-endotoxin efficacy. Antimicrobial efficacy is generally reduced on immobilisation of peptides to a surface, and therefore endotoxin binding efficacy is also expected to be reduced on immobilisation, however, the present inventors have identified that the endotoxin binding efficacy of a peptide comprising the sequence RRP9 (SEQ ID: 1), such as RRP9W4N (SEQ ID: 2) or other sequences defined herein has a substantially maintained endotoxin binding efficacy even when immobilised on the hydrogel. Furthermore, the

As shown in the experimental section, the present inventors have shown surprisingly that attachment of the peptide to a hydrogel substrate does not inhibit anti-endotoxin efficacy. As shown in experiment 2, crosslinked hydrogel granules immobilised with AMPs had similar endotoxin binding efficacy to a corresponding amount of free AMPs in solution.

The hydrogel substrate may be in the form of a crosslinked solid hydrogel as disclosed in for example WO 2019/074422 A1 (Amferia AB). The hydrogel to which the antimicrobial peptide is attached may be in the form of discrete crosslinked amphiphilic hydrogel particles, layers, sheets, or other suitable forms. If the hydrogel is provided as discrete particles, the discrete crosslinked hydrogel particles may be obtained for example via grinding solid crosslinked hydrogel sheets into particles such as described in the experimental section, or other suitable methods of providing discrete hydrogel particles. Discrete crosslinked hydrogel particles may be an especially suitable carrier medium to which the endotoxin neutralising agent may be attached. For example, the particles have a high relative surface area for making the endotoxin neutralising agent available for binding. However, the hydrogel may be in any suitable form to which the endotoxin neutralising particles may be attached. The hydrogel may be a solid sheet with attached AMPs. The hydrogel with attached AMPs may be provided as a wound dressing, ostomy dressing, ostomy baseplate, incision film, a patch, bandage, a plaster, an adhesive, a court plaster, a catheter or a combination thereof. The hydrogel with attached AMPs may be provided as a filter, or a component in a filter for a fluid, such as a patient sample. In particular, for in vitro neutralisation of endotoxins, the hydrogel with attached AMPs may be provided as a component of a filter for a fluid sample.

When used as an endotoxin neutralising filter, the hydrogel with attached AMPs may be subjected to a fluid. The fluid may comprise a known, or unknown amount of endotoxins. Endotoxins present in the fluid are neutralised by the hydrogel with attached AMPs. The fluid may be a patient derived fluid sample, or may be a non-patient derived fluid sample, or may be a combination of a patient derived sample and a non-patient derived reagent. The in vitro filtering/neutralisation of endotoxins may be used for reagents in various processes where endotoxins should not be present. For example, the hydrogel with attached AMPs may be used for filtering reagents and/or cellular material prior to in vitro fertilisation procedures. After contact with the hydrogel with attached AMPs the fluid sample is substantially free from endotoxins.

The process for preparing the discrete hydrogel particles comprises providing a hydrogel composition comprising a first amphiphilic polymer. The first amphiphilic component of the composition may be a crosslinkable amphiphilic polymer. A typical and suitable amphiphilic material is a diacrylate modified poloxamer, such as, polyethylene oxide-polypropylene oxide-polyethylene oxide (DA-PEOₓ-PPO_{y}-PEOₓ-DA, where x and y refer to the number of PEO and PPO groups present respectively) as described in WO 2019/074422 A1. Specifically, the amphiphilic material may be the amphiphilic tri-block co-polymers, polyethylene oxide (100)-polypropylene oxide (70)-polyethylene oxide (100) (Pluronic® F127—BASF Corporation), polyethylene oxide (30)-polypropylene oxide (70)-polyethylene oxide (30) (Pluronic® P123—BASF Corporation).

As stated above, the first amphiphilic component may be a diacrylate derivative of a tri-block copolymer thus enabling the copolymer to be chemically crosslinked. A process for diacrylate modification is provided in WO 2019/074422 A1 (Amferia AB) Experiment 1. The modification may be performed via the reaction of a triblock amphiphilic copolymer with acryloyl chloride to form a diacrylate derivative. Other methods of forming crosslinkable amphiphilic polymers may be possible such as forming methacrylate derivatives or via carboxylic-amine bridges.

The crosslinkable amphiphilic polymer may, in the presence of water, self-assemble to form ordered nanostructures called lyotropic liquid crystals (LLC). In its crosslinked form, that is, after crosslinking, the hydrogel may be considered a chemically crosslinked lyotropic liquid crystal (LLC). The crosslinking of the amphiphilic polymer may be considered to form a polymerized lyotropic liquid crystal (PLLC) having a well-defined structure.

As described above, the crosslinked hydrogel has a repeating and ordered nanostructure. The repeating ordered nanostructure of the amphiphilic hydrogel comprises repeating and alternating hydrophobic-hydrophilic domains. The hydrogel comprises an ordered and repeating nanostructure throughout the hydrogel, that is, not only on the surface of the hydrogel. The crosslinked hydrogel is solid. Intermolecular crosslinking irreversibly locks the ordered structure and results in a hydrogel which has a high integrity and is mechanically resilient.

The covalent attachment of the peptide may be achieved via the covalent bonds between carboxyl groups on the hydrophilic domains of the hydrogel. In the case of the peptide RRP9W4, strong amide bonds are formed between the peptide and the repeating hydrophilic domains of the hydrogel. The peptide may be covalently bonded to the hydrogel via 1-ethyl-3-(3-dimethylaminopropyl) carbodiimide (EDC)-N-hydroxysuccinimide (NHS) activation of the carboxyl groups present on the hydrophilic domains of the hydrogel.

The endotoxin neutralising crosslinked hydrogel may be provided in a dispersion. In particular, if the crosslinked hydrogel is provided in particle form, the endotoxin neutralising crosslinked hydrogel may be provided in a dispersion. The dispersion comprises hydrogel particles as a suspension of particles in a solution. The peptide may be mixed with a solution comprising crosslinked discrete hydrogel particles to covalently attach the peptide to the particles. The solution may be an aqueous solution. The solution may be a saline solution. The solution may be a biocompatible buffer, that is a buffer which is non-toxic to cells such as PBS as shown in the experimental section. As the crosslinked hydrogel particles are amphiphilic the solution may be a non-aqueous solution, such as a non-polar solvent.

The particles in the dispersion are swollen, that is, they have absorbed solution. However, as opposed to other hydrogel dispersions they are truly a suspension of discrete hydrogel particles in a solution, and not simply a liquid hydrogel. A hydrogel may sometimes be described itself as a dispersion or suspension itself, as some hydrogels comprise discrete particles which swell and therein form the hydrogel. In this case the composition comprises a plurality of crosslinked hydrogel particles separate from each other, and separate from the continuous medium, e.g., aqueous solution.

### Experimental Section

### Experiment 1: Endotoxin Binding of AMP Functionalised Crosslinked Hydrogel Discs

### Preparation of Hydrogel Discs

DA-F127 was synthesised as per Experiment 1 in WO 2019/074422 A1 for Pluronic® F127, the DA-F127 having a purity of at least 95% was mixed with water with the composition 30 wt % polymer and 70 wt % water. After mixing, the photoinitiator 2-Hydroxy-4′-(2-hydroxyethoxy)-2-methylpropiophenone was added to the formed gel with an amount corresponding to 0.5 wt % of the polymer in the gel. The gel was placed in a fridge for at least two days. Following cold storage, the gel was cross-linked at 302 nm (UVP Crosslinker; CL-3000M) for 3 minutes (1.5 minutes per side, total does of 0.8-0.9 J/cm²). 12 mm diameter discs were punched into the hydrogel. The now cross-linked hydrogel discs were washed in water for at least two days.

### AMP Binding to Discs

For the AMP functionalization, the hydrogel discs were placed in the bottom of 12 well plates and 2 ml of activation solution was added to the wells, the activation solution consisted of 1 mg/ml of 1-ethyl-3-(3-dimethylaminopropyl) carbodiimide hydrochloride (EDC) and N-hydroxysuccinimide (NHS) in MES buffer, 0.5 M pH~5.5 (adjusted by addition of NaOH). The solutions were left for 30 minutes followed by washing thrice using milli-Q water. In the meantime, the AMP (RRPRPRPRPWWWW-NH2, ≥90% purity, SEQ ID: 2) was weighed into a falcon tube and PBS was added to form a 200 μM solution, this was subsequently added to the activated and washed hydrogel discs, 2 ml per hydrogel, and left to react for 2 h. The discs were then finally washed thrice again using milli-Q water and were ready to be used.

### Preparation of Endotoxin Samples

Control hydrogels and AMP-hydrogel discs were prepared as previously described with roughly a 14 mm diameter (fully swollen). A standard cotton gauze was also cut into 14 mm squares to roughly match the same area and to be used as a standard reference material. The different samples were placed at the bottom of individual 20 ml glass vials, previously autoclaved. P. aeruginosa (CCUG 56489) was inoculated in TSB and incubated until OD 0.55-0.7 which corresponds to around 10⁹ CFU/ml. The bacteria was then diluted to 107 CFU/ml in PBS with TSB added to a concentration of 5%. 50 μl of this solution was then gently placed on top of the different samples as well as just into the glass vial (sample type called PBS hence forth). A 13 mm Ø glass cover slip was then very gently placed on top of the drop, without any pressure, to ensure good and even contact with the samples. The caps of the glass vials were gently screwed on and the vials were left at room temperature undisturbed on a bench to make sure that no liquid flowed away. After 3 or 24 h the glass vials were opened and 4950 μl of PBS were added to the vials. Each vial was then vortexed at max speed for 10 seconds and it was ensured that the glass cover slid separated from the samples. 1 ml of each solution was drawn up with a syringe and passed through a 0.2 μm syringe filter (polyethersulfone, Puradisc 25 from Whatman) into Eppendorf tubes. The tubes from the 3 h samples were frozen until analysis.

### Endotoxin Detection Assay

After the extract from the 24 h samples had been collected, the endotoxin levels of the solutions were analyzed using a ToxinSensor™ Chromogenic LAL Endotoxin Assay Kit from GenScript. The manufacturers protocol was followed and the 3 h samples were diluted 100 times and the 24 h samples were diluted 1000 times before analysis. The data was statistically analyzed using Mann Whitney U-tests.

Results from Endotoxin Detection Assay

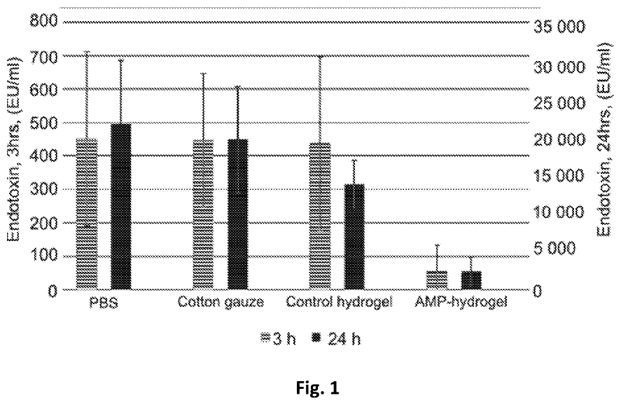

The results from the endotoxin binding results after 3 h and 24 h incubation with P. aeruginosa can be observed in FIG. 1. The PBS, cotton-gauze as well as the control hydrogel all showed similar endotoxin levels at around 450 EU/ml for 3 h of cultivation time, while the AMP-hydrogel samples showed significantly reduced endotoxin levels of 55 EU/ml. The same general trend was observed for 24 h cultivation but at higher levels. The PBS and cotton gauze registered endotoxin levels of around 20 000 EU/ml while the control hydrogel registered slightly lower levels of 13 500 EU/ml. The endotoxin levels observed for the AMP-hydrogels were again significantly reduced at 2 300 EU/ml.

These results clearly show that the endotoxin levels were lower in the solutions that had been in contact with the AMP functionalized hydrogels compared to the other samples. It was also very evident that having bacteria alive and proliferating will over time release more and more endotoxins into the surrounding environment as the endotoxin levels in the PBS samples went from around 450 EU/ml for 3 h to over 20 000 EU/ml for 24 h.

### Experiment 2: Endotoxin Binding of AMP Functionalised Crosslinked Hydrogel Particle Dispersion

### Preparation of Hydrogel Granules

After washing, the hydrogels were grinded to a rough paste using a kitchen blender. The paste was then put in water and an Ultra-Turrax® disperser was used to get a finer particle size and distribution. The obtained solution was then stable for experiments to be performed at convenience.

### AMP Binding to Granules

A known weight of the granules (in swollen form) was obtained by suction filtration and then by putting the granules in a 15 ml falcon tube. This was normally around 2 grams. The resulting batch of granulated particles was divided into two batches. The first batch was the control batch of particles, with no AMP attached. The second batch was activated with the AMP, RRP9W4N. In order to activate the particles, 10 ml of newly prepared EDC/NHS (2 mg/ml) in MES buffer was added to the falcon tube. The tube was sonicated for a few minutes and then put on a shaking plate for 30 minutes. The solution was then suction filtered and washed with water to separate the particles and to wash away the excess EDC/NHS. The granules were then weighed to record any loss and then 10 ml of 400 μM AMP (RRPRPRPRPWWWW-NH2, ≥90% purity, SEQ ID: 2), (solved in PBS) was added to the granules. This was also sonicated quickly and then put on a shaking plate for about 2 hours.

The solution was once again suction filtered and washed with 30 ml of water, but this time the washing solution was recovered in order to measure the amount of peptide still left after activation. The granules are now activated with AMP and can be weighed and put in a solution to get a known concentration for further experiments.

### Preparation of Endotoxin Samples

A colony of E. coli (CCUG 29300) was inoculated in Tryptic Soy Broth, TSB. It was cultivated until mid-log (~10⁹ CFU/ml). The substances to be tested were prepared and diluted to the correct concentrations. As they were subsequently a 2× concentration of interest was prepared. The aim was for 50×MIC for quite fast activity. For this study 2 ml of each (for 2×50×MIC) of the following were prepared:

| Preparation (2 ml) | Description |
|---|---|
| PBS −bac | PBS, no bacteria, control |
| PBS +bac | PBS, bacteria |
| AMP | Free antimicrobial peptide RRP9W4N, |
| | SEQ ID: 2 (300 μM) in PBS |
| Ciprofloxacin | 2 μg/ml Ciprofloxacin |
| Control hydrogel (C-P) | 800 mg of control hydrogel particles (wet) |
| AMP-hydrogel | 800 mg of AMP-hydrogel particles (wet) |

The prepared substances were added to Eppendorf tubes, and 500 μl of PBS was added to each. A solution of 10% TSB, 90% PBS was prepared. 500 μl of the bacterial solution was added to the tubes, except the −bac tube, to prepare a 10⁶ CFU/ml concentration. 500 μl of PBS was added to the −bac tube.

The tubes were vortexed and incubated at 37° C. Each of the samples was removed at desired time intervals (1 hour, 3 hours, and 24 hours).

### Endotoxin Detection Assay

The endotoxin detection protocol was based on the ToxinSensor™ Chromogenic LAL Endotoxin Assay Kit L00350 (Genscript) and the manufacturer's suggested protocol with modifications only based on the concentrations of interest as suggested in the manufacturer's protocol, version 07082022. The absorbance was read on a plate reader at 545 nm using distilled water as a reference. The endotoxin concentrations were measured using a standard curve from the endotoxin standards.

### Results

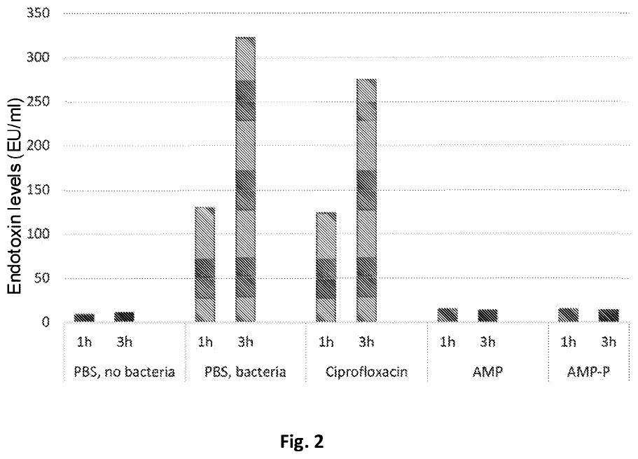

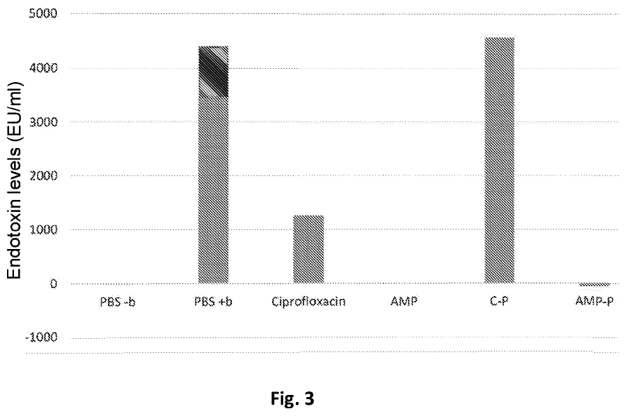

The results of the endotoxin detection assay after 1 hour and 3 hours are shown in FIG. 2. There were no control hydrogel particles present for the 1 hr and 3 hr experiment. The results for 24 hours, including control hydrogel particles are shown in FIG. 3. The baseline for both the 3 hour and 24 hour values was PBS with no bacteria, resulting in a negative value for the hydrogel particles with attached AMPs after 24 hours.

The results show that the antimicrobial peptide neutralised endotoxins in all measured durations of incubation. The results further show that a similar effect was present for the antimicrobial peptide attached to a hydrogel substrate as to the antimicrobial peptide alone. The above results support the use of a peptide comprising the sequences as defined herein as an endotoxin neutralising agent. The peptide may be advantageously attached to a hydrogel substrate for use as an endotoxin neutralising agent. The antibiotic Ciprofloxacin induced significantly more endotoxin release than the free AMP or the AMP attached to hydrogel.

The claimed subject matter is limited only by the accompanying claims. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality.

1-19. (canceled)

20. An amphiphilic peptide for use as an endotoxin neutralising agent, the peptide comprising:
an amino acid sequence having at least 80% sequence identity to the sequence RRP9 (SEQ ID: 1), and
at least four tryptophan residues provided at the C- or N-terminus or therebetween.

21. The amphiphilic peptide for use according to claim 20, wherein the peptide has less than 40 amino acids.

22. The amphiphilic peptide for use according to claim 20, wherein the tryptophan residues are consecutive, or provided in consecutive pairs separated by at most one arginine-proline pair.

23. The amphiphilic peptide for use according to claim 20, wherein the use is in vivo.

24. The amphiphilic peptide for use according to claim 23, wherein the peptide is for use in the treatment of endotoxin induced inflammation.

25. The amphiphilic peptide for use according to claim 20, wherein the peptide comprises an amino acid sequence having at least 90% sequence identity to a sequence selected from RRP9W4N (SEQ ID: 2), RRP7W4RPN (SEQ ID: 4) or RRP5W2RPW2RPN (SEQ ID: 5).

26. The amphiphilic peptide for use according to claim 20, wherein the peptide is RRP9W4N (SEQ ID: 2).

27. The amphiphilic peptide for use according to claim 20, wherein the peptide is attached to a hydrogel substrate.

28. The amphiphilic peptide for use according to claim 27, wherein the peptide is covalently attached to the hydrogel substrate.

29. The amphiphilic peptide for use according to claim 27, wherein the hydrogel substrate is a solid crosslinked hydrogel.

30. The amphiphilic peptide for use according to claim 20, wherein the peptide and/or the hydrogel with the peptide attached is provided on a substrate such as a wound dressing, ostomy dressing, ostomy baseplate, incision film, a patch, bandage, a plaster, an adhesive, a court plaster, a catheter, a filter for a fluid sample, or any combination thereof.

31. A method of endotoxin neutralisation in vitro, the method comprising contacting a fluid sample with an amphiphilic peptide comprising:
an amino acid sequence having at least 80% sequence identity to the sequence RRP9 (SEQ ID: 1), and
at least four tryptophan residues provided at the C- or N-terminus or therebetween.

32. The method of endotoxin neutralisation in vitro according to claim 31, wherein the amphiphilic peptide is covalently attached to a hydrogel substrate.

33. Use of an amphiphilic hydrogel composition in endotoxin neutralisation, the amphiphilic hydrogel in its chemically crosslinked state being a lyotropic liquid crystal and having an ordered nanostructure of hydrophilic and hydrophobic domains, wherein an endotoxin neutralising amphiphilic peptide is covalently attached to the hydrophilic and/or hydrophobic domains.

34. Use of the amphiphilic hydrogel composition according to claim 33, wherein the endotoxin neutralising amphiphilic peptide comprises:
an amino acid sequence having at least 80% sequence identity to the sequence RRP9 (SEQ ID: 1), and
at least four tryptophan residues provided at the C- or N-terminus or therebetween.

35. Use of the amphiphilic hydrogel composition according to claim 34, wherein the amphiphilic peptide has less than 40 amino acids.

36. Use of the amphiphilic hydrogel composition according to claim 34, wherein the peptide comprises an amino acid sequence having at least 90% sequence identity to a sequence selected from RRP9W4N (SEQ ID: 2), RRP7W4RPN (SEQ ID: 4) or RRP5W2RPW2RPN (SEQ ID: 5).

37. Use of the amphiphilic hydrogel composition according to claim 34, wherein the peptide comprises an amino acid sequence having at least 90% sequence identity to the sequence RRP9W4N (SEQ ID: 2).

38. Use of the amphiphilic hydrogel composition according to claim 33, wherein the hydrogel is provided as discrete particles in a dispersion.


SEQUENCE LISTING (6 sequences)

SEQ ID NO: 1 - protein, 9 aa, synthetic construct
RRPRPRPRP

SEQ ID NO: 2 - protein, 13 aa, synthetic construct
  MOD_RES 13: note = AMIDATION
RRPRPRPRPWWWW

SEQ ID NO: 3 - protein, 9 aa, synthetic construct
  MOD_RES 9: note = AMIDATION
RRPRPRPRP

SEQ ID NO: 4 - protein, 13 aa, synthetic construct
  MOD_RES 13: note = AMIDATION
RRPRPRPWWWWRP

SEQ ID NO: 5 - protein, 13 aa, synthetic construct
  MOD_RES 13: note = AMIDATION
RRPRPWWRPWWRP

SEQ ID NO: 6 - protein, 6 aa, synthetic construct
WWRPWW